(19) United States
(12) Patent Application Publication
(10) Pub. No.: US 2025/0339484 A1
(43) Pub. Date: Nov. 6, 2025

(54) COMPOSITION FOR BODY FAT REDUCTION CONTAINING EXTRACTS OF TARAXACUM PLATYCARPUM AND LONICERAE JAPONICA AS ACTIVE INGREDIENTS

(71) Applicant: BOINBIO-CONVERGENCE CO., LTD., Seoul (KR)

(72) Inventors: Woong Mo YANG, Gyeonggi-do (KR); Haesu LEE, Seoul (KR)

(73) Assignee: BOINBIO-CONVERGENCE CO., LTD., Seoul (KR)

(21) Appl. No.: 18/873,449

(22) PCT Filed: Jun. 12, 2023
(86) PCT No.: PCT/KR2023/008023

§ 371 Date: Dec. 10, 2024

(30) Foreign Application Priority Data
Jun. 10, 2022 (KR) 10-2022-0070618
Jun. 12, 2023 (KR) 10-2023-0074772

(51) Int. Cl. A61K 36/288; A61K 36/355; A61P 3/04; A61P 3/06

(52) U.S. Cl. CPC A61K 36/288; A61K 36/355; A61P 3/04; A61P 3/06; A61K 2236/331

(57) ABSTRACT

The present invention relates to a composition for body fat reduction containing extracts of Taraxacum platycarpum and Lonicerae japonica as active ingredients. It has been confirmed that a mixed extract of Taraxacum platycarpum and Lonicerae japonica according to the present invention reduces body weight and body fat when administered to mice with obesity induced by a high-fat diet. Also, it has been confirmed that the mixed extract reduces the size of fat tissue, reduces fat vacuoles (pore) in liver tissue, reduces total cholesterol, triglycerides, and LDL-cholesterol in the blood, and increases HDL-cholesterol. Furthermore, it has been confirmed that the mixed extract suppresses the expression of PPAR-γ which is related to fat synthesis. In addition, it has been confirmed that the mixed extract has a synergistic effect on the regulation of intracellular fat accumulation and fat metabolism factors in cells in which fat accumulation is induced.

### TECHNICAL FIELD

The present disclosure relates to a composition for body fat reduction, including extracts of Taraxacum platycarpum and Lonicerae japonica as active ingredients.

### BACKGROUND ART

Obesity is a condition in which body fat increases due to an imbalance between energy intake and expenditure, and means a condition in which excessive fat is accumulated in the body rather than excessive increase of body weight, and the obesity may cause serious health problems worldwide, such as hypertension, hyperlipidemia, heart disease, diabetes, cancer, and the like. Accordingly, in 1996, in the World Health Organization (WHO), the obesity is classified as a ‘disease’ and recognized as a risk factor for major chronic diseases, and in the Management of Obesity in Korea, 2010 Recommendation, it is clearly indicated that the obesity is a disease and needs to be treated.

This obese population is increasing rapidly worldwide, and the WHO announced that the global obese population is 1.2 billion and is increased to 1.5 billion by 2015. In addition, according to the Organization for Economic Cooperation and Development (OECD) announcement, 31% of the US population and 3.2% of the Korean population are diagnosed as severely obese with BMI of 30 kg/m² or higher.

However, the biggest problem with current obesity treatment may be drug preference, and unproven drugs are used, or the drugs are abused even when drug treatment is not required, which is problematic. In particular, the use of psychotropic antiobestic drugs should be limited to adjunctive therapy in obesity treatment for only a few weeks, but there is a major problem in that there is no active management for the psychotropic antiobestic drugs. In particular, as serious side effects such as increased blood pressure, increased respiration, impaired consciousness, and hypothyroid were reported for a sibutramine ingredient, an appetite suppressant that accounted for half of domestic antiobestic drugs, the sales thereof were halted even in Korea in 2010, following Europe and the United States. Thereafter, as the supply of a phendimetrazine ingredient has increased, the supply of antiobestic drugs has be maintained at a normal level, but the side effects that appear after taking still remain a major problem.

In addition to the drug therapy, body weight control methods for obesity treatment include diet therapy, exercise therapy, behavior modification therapy, and the like. The diet-related industry has been formed across various fields, including formulated foods for controlling a body weight, diet health functional foods, health centers, and obesity clinics. In particular, the domestic diet food market scale is approximately 150 to 200 billion won, growing by 7 to 10% annually. Recently, as serious side effects of antiobestic drug have become known, interest is focusing on foods using natural products that are easier to access and have fewer side effects than pharmaceutical drugs. Among representative health functional foods for body weight control, conjugated linolenic acid (CLA) is a double-bond conjugated isomer of linoleic acid. Through various experiments, it has been reported effects including reducing fat absorption and suppressing fat accumulation by inhibiting lipoprotein enzymes, increasing basal metabolic rate by increasing mitochondrial activity, and reducing the number of fat cells by increasing apoptosis of fat cells. Through clinical trials, it was found an effect of reducing the body weight to average 2.5 kg and body fat by 15 to 20% after 3 months when a person takes about 3 g per day. However, such a body weight reduction effect is not consistently shown, and it has been reported the result that the CLA may increase insulin resistance and cause fatty liver, and thus caution is required to administer complex linoleic acid products to patients with increased insulin resistance, including type 2 diabetes patients.

Among health functional foods that have been taken for obesity treatment, there is none that is more effective than drugs that have been currently prescribed for obesity treatment. In addition, there is a large price difference between products, and false advertising regarding functionality confuses consumers when selecting a product, and when considering a therapeutic effect, it is not cheap. Foods that have been argued to have a body weight reduction effect may show the body weight reduction effect in conjunction with regular exercise and dietary control by the consumer, but the market for products with unproven efficacy is highly formed due to vague expectations about the efficacy of foods, and thus it is urgent to develop foods derived from natural products whose efficacy may be objectively proven is urgent.

Meanwhile, Taraxacum platycarpum is a dried medicinal herb of the whole plant of Taraxacum platycarpum H. Dahlstedt or plants belonging to the same genus, and has been reported to have antibacterial, immune function enhancement, bile secretion, liver function protection, and diuretic effects. The appearance has a long spindle-shaped root and several long oval-shaped, wing-shaped leaves attached to the root, and has yellow-green or gray-green outer surfaces of the leaves, light brown or black brown roots, and some flowers and fruits.

Lonicerae japonica is dried stem and leaves of Lonicerae japonica Thunb, a plant in the Lonicera family, and grows wild on the foothills of mountains and the field dikes in various regions. The Lonicerae japonica is prepared by harvesting the stems between late summer and fall and drying them in the sun and has a sweet taste and a cool nature. The Lonicerae japonica is known to have an antibacterial effect against Staphylococcus albus and the like, and is prescribed for colds, fever, body aches, dermatitis, boils, dysentery, and pain in the tetraplegia limbs.

Accordingly, the present inventors confirmed that a mixed extract of Taraxacum platycarpum and Lonicerae japonica has an excellent obesity improvement effect, and thus completed the present disclosure.

### DISCLOSURE

### Technical Problem

An object of the present disclosure is to provide a food composition for preventing or improving obesity, including a mixed extract of Taraxacum platycarpum and Lonicerae japonica as an active ingredient.

Another object of the present disclosure is to provide a pharmaceutical composition for preventing or treating obesity, including a mixed extract of Taraxacum platycarpum and Lonicerae japonica as an active ingredient.

Yet another object of the present disclosure is to provide a food composition for body fat reduction, including a mixed extract of Taraxacum platycarpum and Lonicerae japonica as an active ingredient.

Yet another object of the present disclosure is to provide a pharmaceutical composition for body fat reduction, including a mixed extract of Taraxacum platycarpum and Lonicerae japonica as an active ingredient

Yet another object of the present disclosure is to provide a method for treating obesity, including administering to a subject a mixed extract of Taraxacum platycarpum and Lonicerae japonica in a pharmaceutically effective amount.

### Technical Solution

In order to achieve the above aspects, the present disclosure provides a food composition for preventing or improving obesity, including a mixed extract of Taraxacum platycarpum and Lonicerae japonica as an active ingredient.

Further, the present disclosure provides a pharmaceutical composition for preventing or treating obesity, including a mixed extract of Taraxacum platycarpum and Lonicerae japonica as an active ingredient.

Further, the present disclosure provides a food composition for body fat reduction, including a mixed extract of Taraxacum platycarpum and Lonicerae japonica as an active ingredient.

Further, the present disclosure provides a pharmaceutical composition for body fat reduction, including a mixed extract of Taraxacum platycarpum and Lonicerae japonica as an active ingredient.

Further, the present disclosure provides a method for treating obesity, including administering to a subject a mixed extract of Taraxacum platycarpum and Lonicerae japonica in a pharmaceutically effective amount.

### Advantageous Effects

According to the present disclosure as described above, it has been confirmed that a mixed extract of Taraxacum platycarpum and Lonicerae japonica reduces body weight and body fat when administered to mice with obesity induced by a high-fat diet. Also, it has been confirmed that the mixed extract reduces the size of fat tissue, reduces fat vacuoles (pore) in liver tissue, reduces total cholesterol, triglycerides, and LDL-cholesterol in the blood, and increases HDL-cholesterol. Furthermore, it has been confirmed that the mixed extract suppresses the expression of PPAR-γ which is related to fat synthesis. In addition, it has been confirmed that the mixed extract has a synergistic effect on the regulation of intracellular fat accumulation and fat metabolism factors in cells in which fat accumulation is induced and thus the mixed extract can be usefully used in related industries.

### DESCRIPTION OF DRAWINGS

FIG. 1 is a diagram quantifying changes in body weight of mice according to administration of a mixed extract of Taraxacum platycarpum and Lonicerae japonica of the present disclosure.
ND: Normal diet group (normal control group)
HFD: High-fat diet group (obese control group/disease control group)
ORL: High-fat diet+Orlistat administered group (positive control group)
FORMULA: High-fat diet+mixed extract administered group of Taraxacum platycarpum and Lonicerae japonica (experimental group)

FIG. 2 is a diagram showing the analysis of body fat of mice using Dual-energy X-ray absorptiometry (DXA) according to administration of a mixed extract of Taraxacum platycarpum and Lonicerae japonica of the present disclosure.

FIG. 3 is a diagram quantifying body fat mass and body fat percentage of mice according to administration of a mixed extract of Taraxacum platycarpum and Lonicerae japonica of the present disclosure (A: quantification of body fat mass, B: quantification of body fat percentage).

FIG. 4 is a diagram confirming the size of fat cells in mouse fat tissue according to administration of a mixed extract of Taraxacum platycarpum and Lonicerae japonica according to the present disclosure (A: confirmation of fat cells, B: quantification of fat cell diameter).

FIG. 5 is a diagram confirming the distribution of fat vacuoles (pore) in mouse liver tissue according to administration of a mixed extract of Taraxacum platycarpum and Lonicerae japonica according to the present disclosure.

FIG. 6 is a diagram analyzing the lipid content in the blood of mice according to administration of a mixed extract of Taraxacum platycarpum and Lonicerae japonica of the present disclosure (A: total cholesterol content, B: triglyceride content, C: HDL-cholesterol content, D: LDL-cholesterol content).

FIG. 7 is a diagram showing RT-PCR analysis of the expression of Peroxisome proliferator-activated receptor-gamma (PPAR-γ), as a fat synthesis factor in mouse fat tissue according to administration of a mixed extract of Taraxacum platycarpum and Lonicerae japonica of the present disclosure (A: PT-PCR results, B: PPAR-γ expression quantification).

FIG. 8 is a diagram showing an effect of suppressing intracellular fat accumulation of each extract and a mixed extract of Taraxacum platycarpum and Lonicerae japonica of the present disclosure, confirmed by oil red O staining (A: staining results, B: quantification of staining results).

FIG. 9 is a diagram showing the expression of fat differentiation inhibition and metabolism inhibition factors of each extract and a mixed extract of Taraxacum platycarpum and Lonicerae japonica of the present disclosure, confirmed by Western blot (A: ATGL results and quantification, B: p-AMPK results and quantification).

FIG. 10 is a diagram showing the expression inhibition of fat differentiation and fat accumulation factors by each extract and a mixed extract of Taraxacum platycarpum and Lonicerae japonica of the present disclosure, confirmed by RT-PCR (A: LPL results and quantification, B: SREBP-1c results and quantification).

### BEST MODE OF THE INVENTION

Hereinafter, exemplary embodiments of the present disclosure will be described in detail with reference to the accompanying drawings. In the following description, detailed descriptions of techniques well-known to those skilled in the art may be omitted. Further, in describing the present disclosure, the detailed description of associated known functions or constitutions will be omitted if it is determined to unnecessarily make the gist of the present disclosure unclear. In addition, terminologies used in the present disclosure are terminologies used to properly express preferred exemplary embodiments of the present disclosure, which may vary according to a user, an operator's intention, or customs in the art to which the present disclosure pertains.

Accordingly, definitions of the terminologies need to be described based on contents throughout this specification. Throughout this specification, unless explicitly described to the contrary, when a certain part “comprises” a certain component, it will be understood to imply the inclusion of stated elements, not the exclusion of any other elements.

The present disclosure provides a food composition for preventing or improving obesity, including a mixed extract of Taraxacum platycarpum and Lonicerae japonica as an active ingredient.

As used in the present disclosure, the term “prevention” refers to any action that suppresses the symptoms of a specific disease or delays its progression by administering the composition of the present disclosure.

As used in the present disclosure, the “improvement” means all actions that at least reduce parameters associated with conditions to be treated, such as the severity of symptoms.

In addition to containing the active ingredient of the present disclosure, the food composition of the present disclosure may contain various flavoring agents, natural carbohydrates, or the like as an additional ingredient, like conventional food compositions.

Examples of the above-described natural carbohydrates include conventional sugars, including monosaccharides, such as glucose, fructose, etc.; disaccharides, such as maltose, sucrose, etc.; and polysaccharides, such as dextrin, cyclodextrin, etc., and sugar alcohols such as xylitol, sorbitol, erythritol, etc. The above-described flavoring agents may be advantageously used with natural flavoring agents (thaumatin), stevia extracts (e.g., rebaudioside A, glycyrrhizin, etc.), and synthetic flavoring agents (saccharin, aspartame, etc.). The food composition of the present disclosure may be formulated in the same manner as the pharmaceutical composition to be used as a functional food or added to various foods. The foods capable of adding the composition of the present disclosure include, for example, beverages, meat, chocolate, foods, confectionery, pizza, ramen, other noodles, gums, candies, ice creams, alcohol beverages, vitamin complexes, health food supplements, etc.

In addition, the food composition may contain various nutrients, vitamins, minerals (electrolytes), flavoring agents such as synthetic and natural flavoring agents, coloring agents and enhancers (cheese, chocolate, etc.), pectic acid and salts thereof, alginic acid and salts thereof, organic acid, a protective colloidal thickener, a pH adjusting agent, a stabilizer, a preservative, glycerin, alcohol, a carbonic acid agent used in a carbonated drink, and the like, in addition to the extract as the active ingredient. In addition, the food composition of the present disclosure may contain pulps for preparing natural fruit juice, fruit juice beverages, and vegetable beverages.

The functional food composition of the present disclosure may be prepared and processed in the form of tablets, capsules, powders, granules, liquids, pills, etc., for the purpose of prevention or treatment of obesity. In the present disclosure, the ‘health functional food composition’ refers to foods prepared and processed by using raw materials or ingredients with functionality, which are useful for the human body according to the Art No. 6727 on Health Functional Food, and means foods taken for adjusting nutrients for the structures and functions of the human body or obtaining a useful effect on health applications such as physiological actions. The health functional food of the present disclosure may include conventional food additives, and the suitability as the food additives is determined by the specifications and standards for the corresponding item in accordance with the general rules of the Food Additives Codex, general test methods, etc., that are approved by the Food and Drug Administration, unless otherwise specified. The items disclosed in the ‘Food Additives Codex’ may include, for example, chemical composites such as ketones, glycine, calcium citrate, nicotinic acid, cinnamic acid, etc.; natural additives such as persimmon color, licorice extract, crystal cellulose, Kaoliang color, guar gum, etc.; mixed formulations such as sodium L-glutamic acid formulations, noodle additive alkali agents, preservative formulations, tar color formulations, etc. For example, the health functional food in the form of tablets may be prepared by granulating a mixture obtained by mixing the active ingredient of the present disclosure with an excipient, a binder, a disintegrant, and other additives, and then compression-molding the mixture by adding a slip modifier and the like, or directly compression-molding the mixture. In addition, the health functional food in the form of tablets may also contain a flavors enhancer or the like as needed. In the health functional food in the form of capsules, hard capsules may be prepared by filling a mixture mixed with the active ingredient of the present disclosure and additives such as excipients into conventional hard capsules, and soft capsules may be prepared by filling a mixture mixed with the active ingredient of the present disclosure and additives such as excipients into capsule bases such as gelatin. The soft capsules may contain a plasticizer such as glycerin or sorbitol, a colorant, a preservative, and the like, if necessary. The health functional food in the form of pills may be prepared by molding a mixture obtained by mixing the active ingredient of the present disclosure with an excipient, a binder, a disintegrant, etc. by conventional known methods, and may also be coated with white sugar or other coating agents or surface-coated with materials such as starch and talc, if necessary. The health functional food in the form of granules may be prepared by granulizing a mixture obtained by mixing the active ingredient of the present disclosure with an excipient, a binder, a disintegrant, etc. by conventional known methods and may contain a flavoring agent, a flavors enhancer, etc., if necessary.

According to an exemplary embodiment of the present disclosure, the Taraxacum platycarpum and the Lonicerae japonica may be mixed and extracted in a weight ratio of 1 to 100:1 to 100, preferably in a weight ratio of 1:1.5, but is not limited thereto.

According to an exemplary embodiment of the present disclosure, the Taraxacum platycarpum and the Lonicerae japonica may be extracted with a solvent selected from the group consisting of water, lower alcohols having C1 to C4, a lower alcohol aqueous solution, hexane, chloroform, ethyl acetate, and acetone.

According to an exemplary embodiment of the present disclosure, the Taraxacum platycarpum and the Lonicerae japonica may be mixed with the solvent and extracted in a weight ratio of 1 to 100:1 to 100.

According to an exemplary embodiment of the present disclosure, the mixed extract may reduce body weight, and the body weight reduction may mean reducing body fat, body fat mass, or body fat percentage.

The “body fat” of the present disclosure means the amount of fat in the body. The body fat includes not only subcutaneous fat or visceral fat, but also all the fat in the blood and lipids in cell membranes.

The “body fat mass” in the present disclosure means the amount of fat (kg) in the body.

The “body fat percentage” of the present disclosure refers to a ratio of body fat to body weight, and the normal body fat percentage is 10 to 20% for men and 18 to 28% for women. The ratio of visceral fat to subcutaneous fat varies greatly from person to person depending on the degree of obesity and the amount of exercise. When adults become obese, it is often because visceral fat becomes excessive and the abdominal fat percentage increases.

According to an exemplary embodiment of the present disclosure, the mixed extract may reduce the size of fat cells in fat tissue and inhibit the formation of fat vacuoles (pore) in liver tissue.

According to an exemplary embodiment of the present disclosure, the mixed extract may reduce the content of lipids selected from the group consisting of total cholesterol, triglyceride, low density lipoprotein cholesterol (LDL-cholesterol), and very low density lipoprotein cholesterol (VLDL-cholesterol) in the blood.

According to an exemplary embodiment of the present disclosure, the mixed extract may increase high density lipoprotein cholesterol (HDL-cholesterol) in the blood.

According to an exemplary embodiment of the present disclosure, the mixed extract may inhibit the expression of a fat synthesis factor, PPAR-γ (Peroxisome proliferator-activated receptor-gamma).

The “PPAR-γ (Peroxisome proliferator-activated receptor-gamma)” of the present disclosure is one of three isoforms of a peroxisome proliferator-activated receptor, which is a type of nuclear receptor. The peroxisome proliferator-activated receptor is involved in the regulation of cell differentiation, growth, and metabolism, and all three isoforms of these peroxisome proliferator-activated receptors form heterodimers with another nuclear orphan receptor called RXR to bind to transcriptional regulatory sites of genes. The three isoforms of PPAR are mainly expressed in different tissues and the functions thereof also vary depending on a tissue. PPARα is a PPAR mainly expressed in the liver and is a representative transcription factor that regulates gluconeogenesis, which promotes glucose synthesis in the liver during fasting, and also serves to promote β-oxidation of fatty acids in the liver. PPARβ/δ is a PPAR expressed in various tissues and plays a very important role in lipid metabolism, and particularly regulates the expression of various genes related to β-oxidation of fatty acids. PPARγ is a transcription factor mainly expressed in fat tissue and is the most important factor in the differentiation of fat tissue. As the function of PPARγ is activated, the amount of fat tissue increases in the body, inducing obesity or overweight. For example, thiazolidinedione (TZD), a well-known therapeutic agent for type 2 diabetes, is a synthetic agonist that activates the function of PPARγ and has an excellent effect of treating insulin resistance, but has side effects of causing body weight gain, which is because activated PPARγ promotes fat differentiation in the body to increase the body fat mass.

According to an exemplary embodiment of the present disclosure, the mixed extract may regulate the expression of fat metabolism factors, and the fat metabolism factors may be selected from the group consisting of Adipose triglyceride lipase (ATGL), phosphorylation-AMP-activated protein kinase (p-AMPK), lipoprotein lipase (LPL), and sterol regulatory element-binding protein-1c (SREBP-1c).

According to an exemplary embodiment of the present disclosure, the regulating of the expression of the fat metabolism factors may be increasing the expression of ATGL or p-AMPK.

The “ATGL (Adipose triglyceride lipase)” of the present disclosure is an enzyme that catalyzes the first reaction of lipolysis, which hydrolyzes triacylglycerol into diacylglycerol. The ATGL affects the later stage of lipolysis by changing the concentration of diacylglycerol and serves to control lipolysis.

The “p-AMPK (phosphorylation-AMP-activated protein kinase)” of the present disclosure is an active type of AMPK, and the activated AMPK is an enzyme that stimulates liver fatty acid oxidation, stimulates skeletal muscle fatty acid oxidation and glucose uptake, inhibits cholesterol synthesis, inhibits lipogenesis and triglyceride synthesis, inhibits fat cell lipogenesis, and inhibits fat cell differentiation, thereby inhibiting lipid synthesis and fat cell differentiation.

According to an exemplary embodiment of the present disclosure, the regulating of the expression of the fat metabolism factors may be reducing the expression of LPL or SREBP-1c.

The “LPL (lipoprotein lipase)” of the present disclosure is a member of the lipase gene family, and is an enzyme that exists mainly in the inner walls of capillaries of fat tissue, muscles, and the like, and hydrolyzes triacylglycerol in plasma lipoproteins rich in triacylglycerols derived from the intestine or liver. The most important regulatory factors in the process of fat cell formation are known as peroxisome proliferator-activated receptors (PPARs) and CAAT/enhancer binding proteins (C/EBPs). When differentiation begins, initial transcription factors C/EBP-β and C/EBP-δ increase the expression levels of C/EBP-α and PPAR-γ, which ultimately increase the expression of final markers such as lipoprotein lipase (LPL), leptin, adiponectin, and fatty acid binding protein (FABP) 4, and thus are used as markers of fat differentiation.

The “SREBP-1c (sterol regulatory element-binding protein-1c)” of the present disclosure is a factor that induces lipogenesis, and when mTORC1 is activated by insulin, the production of SREBP-1c increases, and fatty acids are stored as triglycerides, thereby increasing fat accumulation in fat cells.

Further, the present disclosure provides a pharmaceutical composition for preventing or treating obesity, including a mixed extract of Taraxacum platycarpum and Lonicerae japonica as an active ingredient.

As used in the present disclosure, the term “treatment” refers to all actions that improve or beneficially change the symptoms of a specific disease by administering the composition of the present disclosure.

The pharmaceutical composition of the present disclosure may further include an adjuvant in addition to the active ingredient. The adjuvant may be used with any adjuvant known in the art without limitation, but further include, for example, a Freund's complete adjuvant or an incomplete adjuvant to increase the effect thereof.

The pharmaceutical composition according to the present disclosure may be prepared in the form of incorporating the active ingredient into a pharmaceutically acceptable carrier. Here, the pharmaceutically acceptable carrier includes carriers, excipients and diluents commonly used in a pharmaceutical field. The pharmaceutically acceptable carrier that may be used in the pharmaceutical composition of the present disclosure is not limited thereto, but may include lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinylpyrrolidone, water, methyl hydroxybenzoate, propyl hydroxybenzoate, talc, magnesium stearate, and mineral oil.

The pharmaceutical composition of the present disclosure may be formulated and used in the form of oral formulations, such as powders, granules, tablets, capsules, suspensions, emulsions, syrups, aerosols, etc., external preparations, suppositories, or sterile injectable solutions according to each conventional method.

The formulations may be prepared by using diluents or excipients, such as a filler, an extender, a binder, a wetting agent, a disintegrating agent, a surfactant, etc., which are generally used. Solid formulations for oral administration include tablets, pills, powders, granules, capsules, etc., and these solid formulations may be prepared by mixing at least one or more excipients, for example, starch, calcium carbonate, sucrose, lactose, gelatin, etc. with the active ingredient. Further, lubricants such as magnesium stearate and talc may be used in addition to simple excipients. Liquid formulations for oral administration may correspond to suspensions, oral liquids, emulsions, syrups, etc., and may include various excipients, for example, a wetting agent, a sweetener, an aromatic agent, a preserving agent, etc., in addition to the commonly used diluents, such as water and liquid paraffin. Formulations for parenteral administration include sterile aqueous solutions, non-aqueous solvents, suspensions, emulsions, lyophilized agents, and suppositories. As the non-aqueous solution and the suspension, propylene glycol, polyethylene glycol, vegetable oil such as olive oil, injectable ester such as ethyl oleate, etc. may be used. As the base material of the suppository, witepsol, Tween 61, cacao butter, laurinum, glycerogelatin, etc. may be used.

The pharmaceutical composition according to the present disclosure may be administered to a subject through various routes. All methods of administration may be expected, and the pharmaceutical composition may be administered by, for example, oral, intravenous, intramuscular, subcutaneous, and intraperitoneal injection.

The dose of the pharmaceutical composition according to the present disclosure is selected in consideration of the age, body weight, sex, physical conditions, and the like of a subject. It is obvious that the concentration of the active ingredient included in the pharmaceutical composition may be variously selected according to a subject, and preferably included in the pharmaceutical composition at a concentration of 0.01 to 5,000 μg/ml. When the concentration is less than 0.01 μg/ml, pharmaceutical activity may not be exhibited, and when the concentration exceeds 5,000 μg/ml, toxicity to the human body may be exhibited.

Further, the present disclosure relates to a food composition for body fat reduction, including a mixed extract of Taraxacum platycarpum and Lonicerae japonica as an active ingredient.

Further, the present disclosure relates to a pharmaceutical composition for body fat reduction, including a mixed extract of Taraxacum platycarpum and Lonicerae japonica as an active ingredient.

Further, the present disclosure provides a method for treating obesity, including administering to a subject a mixed extract of Taraxacum platycarpum and Lonicerae japonica in a pharmaceutically effective amount.

The treatment method of the present disclosure includes administering to a subject the mixed extract of Taraxacum platycarpum and Lonicerae japonica in a therapeutically effective amount. It is preferred that a specific therapeutically effective amount for a specific subject is differently applied depending on various factors including the type and degree of a response to be achieved, a specific composition including whether other agents are used in some cases, the age, body weight, general health conditions, sex, and diet of a subject, an administration time, an administration route, a secretion rate of the composition, a duration of treatment, and a drug used in combination or simultaneously with the specific composition, and similar factors well known in the medical field. A daily dose may be 0.0001 to 100 mg/kg, preferably 0.01 to 100 mg/kg, based on the amount of the pharmaceutical composition of the present disclosure, and may be administered 1 to 6 times a day. However, it is obvious to those skilled in the art that the dosage or dose of each active ingredient does not cause side effects by including the content of each active ingredient too high. Therefore, the effective amount of the composition suitable for the purpose of the present disclosure is preferably determined in consideration of the aforementioned matters.

The subject is applicable to any mammal, and the mammal includes not only humans and primates, but also livestock such as cattle, pig, sheep, horse, dog, and cat.

### MODES OF THE INVENTION

Hereinafter, the present disclosure will be described in more detail through Examples. These Examples are to explain the present disclosure in more detail, and it will be apparent to those skilled in the art that the scope of the present disclosure is not limited to these Examples.

### Preparation Example 1

### Preparation of Mixed Extract of Taraxacum platycarpum and Lonicerae japonica

A mixed extract (Formula) of Taraxacum platycarpum and Lonicerae japonica of the present disclosure was prepared. Specifically, the Taraxacum platycarpum and Lonicerae japonica were purchased from Dongyang Herb (Seoul, Korea). 100 g of Taraxacum platycarpum and 150 g of Lonicerae japonica were mixed, placed in distilled water, and reflux-extracted for 1 hour. Thereafter, the extracted solution was filtered using filter paper, and the obtained filtrate was concentrated at 60° C. using a rotary evaporator and then spray-dried. The obtained mixed extract (Formula or TL-700) of Taraxacum platycarpum and Lonicerae japonica was stored at −20° C.

### Example 1

### Confirmation of Body Weight Reduction Effect

A body weight reduction effect of a mixed extract of Taraxacum platycarpum and Lonicerae japonica of the present disclosure was confirmed. Specifically, 5-week-old male C57BL/6J mice having 19 to 21 g of body weight were bred under a 12-h light/dark cycle, room temperature of 22±2° C., and humidity of 50±5%. After acclimation for 7 days by freely feeding food and water, a high-fat diet (HFD, Research Diets, D12492) containing 60% fat was fed for 14 weeks to induce obesity. Simultaneously with the high-fat diet treatment, the mixed extract (Formula) was orally administered at a concentration of 100 mg/kg by 100 μL 5 times a week for 14 weeks, for a total of 70 times. As control group mice, a normal diet group (ND) and a high-fat diet (HFD) group orally administered with the same amount (100 μL) of distilled water as the high-fat diet were used. Orlistat known as an antiobestic drug was used as a positive control group. The body weight reduction effect was confirmed by measuring body weight at the end of the experiment.

As a result, as shown in FIG. 1, the body weight of the high-fat diet group (HFD) was 40.52±2.092 g, whereas the body weight of the Formula group administered with the mixed extract was 33.35±1.410 g, which was decreased by 17.69% compared to the high-fat diet group.

### Example 2

### Confirmation of Body Fat Reduction Effect

A body weight reduction effect of a mixed extract of Taraxacum platycarpum and Lonicerae japonica of the present disclosure was confirmed. Specifically, the mice of Example 1 were anesthetized at the end of the experiment, and body fat was measured using an X-ray body composition analyzer (dual-energy X-ray absorptiometry, DXA).

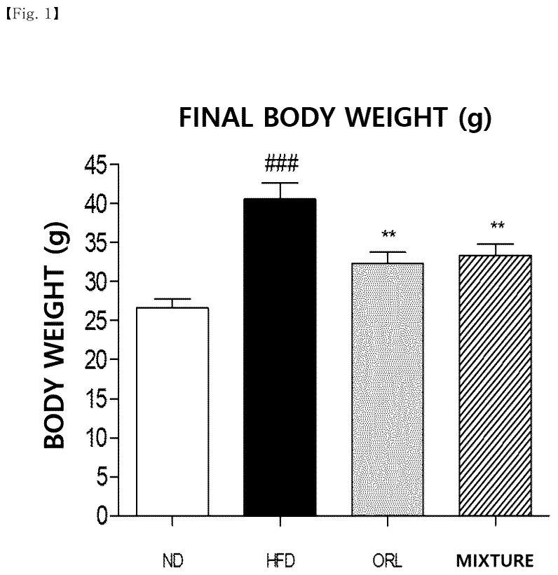

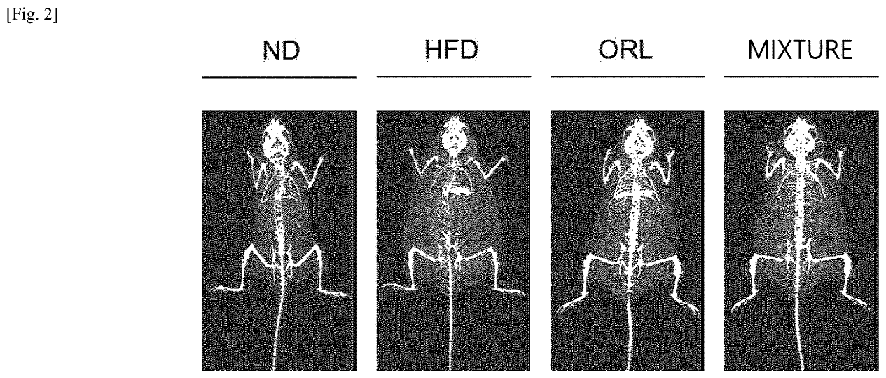

As a result, as shown in FIG. 2, the body fat (red) of the high-fat diet group significantly increased compared to the ND group taking the normal diet, but the Formula group administered with the mixed extract showed a decrease in the body fat increased by the high-fat diet.

### Example 3

### Confirmation of Effects of Reducing Body Fat Mass and Body Fat Percentage

The effects of reducing body fat mass and body fat percentage of a mixed extract of Taraxacum platycarpum and Lonicerae japonica of the present disclosure were confirmed. Specifically, the body fat mass and the body fat percentage of mice were compared using DXA in the same manner as in Example 3 above.

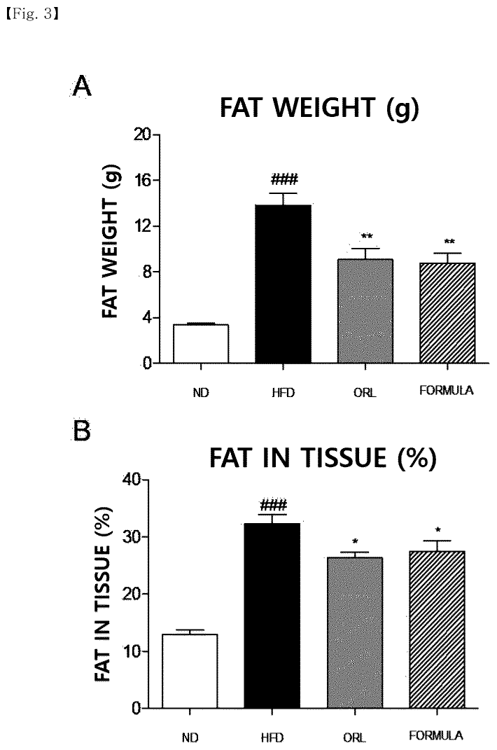

As a result, the body fat weight of the high-fat diet group (HFD) was 13.81±1.025 g, but in the Formula group administered with the mixed extract, the body fat weight was 8.775±0.851 g, which showed a 36.46% decrease in body fat mass compared to the high-fat diet group (FIG. 3A). In addition, the body fat percentage of the high-fat diet group was 32.36%, but in the Formula group, the body fat percentage was 27.47%, which showed a 15.11% decrease compared to the high-fat diet group (FIG. 3B).

### Example 4

### Histological Evaluation

### <4-1> Histological Evaluation of Fat Tissue

In order to confirm the effect of the mixed extract of Taraxacum platycarpum and Lonicerae japonica of the present disclosure, histological evaluation of fat tissue was performed. Specifically, the mice of Example 1 were humanely sacrificed at the end of the experiment, and the fat tissue around the epididymis was obtained. The obtained fat tissue was then fixed in 10% paraformaldehyde for 24 hours and washed to sufficiently remove a fixing solution that had penetrated the tissue. Thereafter, the moisture in the tissue was removed through a dehydration process sequentially with alcohol concentrations of 70%, 90%, 95%, and 100%, and then a paraffin block was produced using xylene as a transparent agent. The prepared paraffin block was sectioned at 5 μm intervals by microtome cutting, deparaffinized and hydrated, and then staining with hematoxylin & eosin (H&E) solution. The stained slide was observed under an optical microscope at 400× magnification, and the diameter of fat cells was measured using the Image J program to analyze the size of fat tissue.

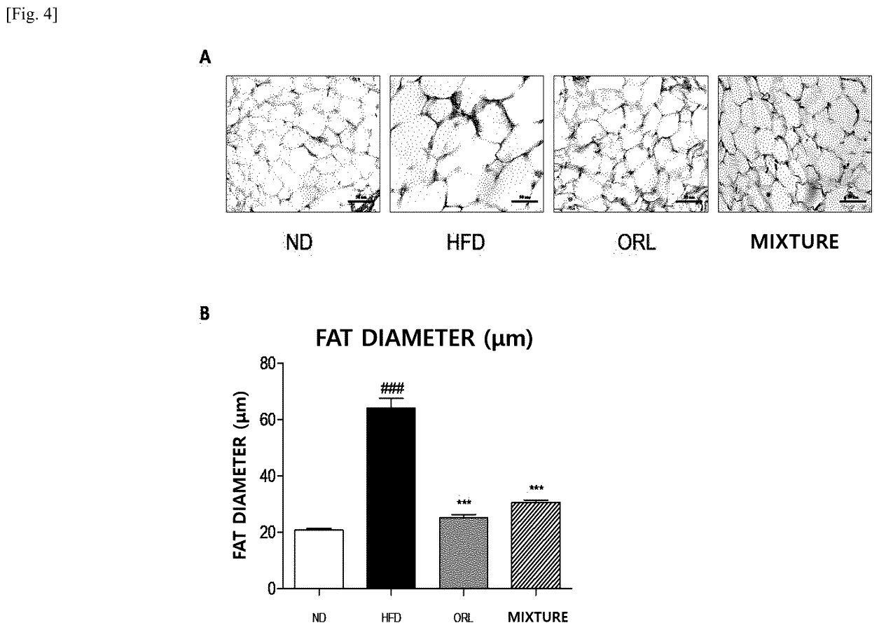

As a result, as shown in FIG. 4, in the high-fat diet group (HFD), the diameter of fat cells in the fat tissue was 64±3.42 μm, but in the Formula group administered with the mixed extract, the diameter was 30.61±0.89 μm, which showed a 52.17% decrease compared to the high-fat diet group.

### <4-2> Confirmation of Suppression of Fat Vacuoles (Pore) in Liver Tissue

In order to confirm the effect of the mixed extract of Taraxacum platycarpum and Lonicerae japonica of the present disclosure, an effect of suppressing fat vacuoles (pore) in liver tissue was confirmed by a histological method. Specifically, the effect was confirmed using the same method as Example 4-1 above.

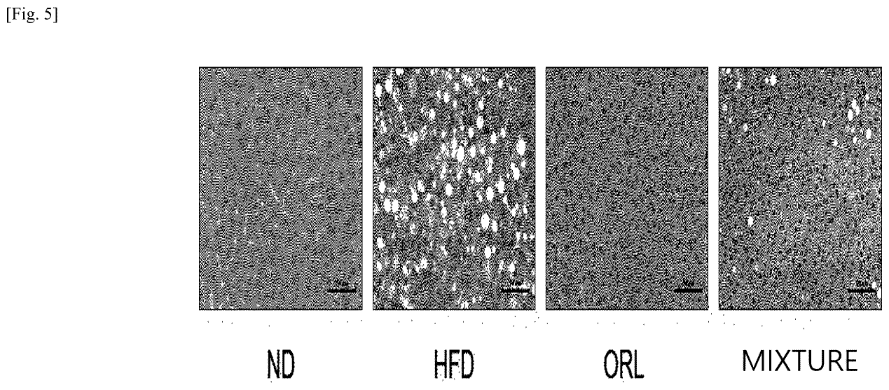

As a result, as shown in FIG. 5, in the liver tissue of the high-fat diet group (HFD), a plurality of fat vacuoles (pore) increased compared to the normal diet group (ND), but in the Formula group administered with the mixed extract, the number of vacuoles (pore) increased by the high-fat diet was decreased.

### Example 5

### Content Analysis of Blood Cholesterol and Neutral Fat

It was confirmed whether the mixed extract of Taraxacum platycarpum and Lonicerae japonica of the present disclosure reduced blood cholesterol and neutral fat. Specifically, the blood was collected through the orbital vein from the mice sacrificed in Example 4 above. Thereafter, the collected blood was centrifuged at 17,000 rpm at 4° C. for 20 minutes to isolate the serum. To measure the contents of total cholesterol, triglycerides, high density lipoprotein cholesterol (HDL-cholesterol), low density lipoprotein cholesterol (LDL-cholesterol)/very low density lipoprotein cholesterol (VLDL-cholesterol) in the isolated serum, the blood cholesterol and triglyceride contents were analyzed using the HDL and LDL/VLDL Cholesterol Assay Kit and the Triglyceride Quantification Assay Kit according to each protocol.

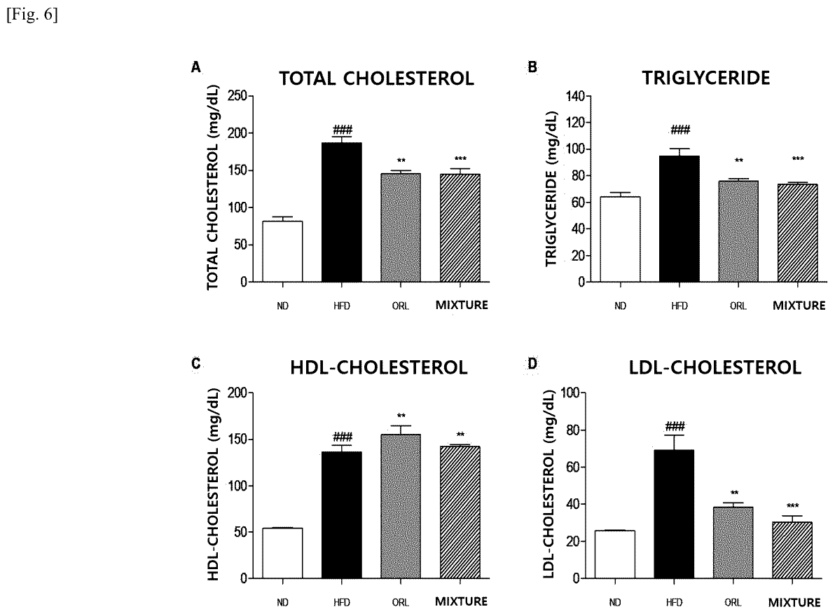

As a result, as shown in FIG. 6, in the Formula group administered with the mixed extract of the present disclosure, the total cholesterol, triglycerides, and LDL-cholesterol contents were all significantly decreased compared to the high-fat diet group (HFD), and the HDL-cholesterol content was significantly increased. More specifically, the contents of total cholesterol, triglycerides, HDL-cholesterol, and LDL-cholesterol in the high-fat diet (HFD) group were 186.5 mg/dL, 94.75 mg/dL, 136.42 mg/dL, and 69.22 mg/dL, respectively, but in the Formula group, the contents were 144.77 mg/dL, 73.43 mg/dL, 142.09 mg/dL, and 30.28 mg/dL, respectively. Compared to the high-fat diet (HFD) group, the contents of total cholesterol, triglycerides, and LDL-cholesterol in the Formula group decreased by 22.36%, 22.5%, and 56.26%, while the HDL-cholesterol content increased by 4.18%.

### Example 6

### Confirmation of Suppression of Expression of Fat Synthesis Factors

It was confirmed whether the mixed extract of Taraxacum platycarpum and Lonicerae japonica of the present disclosure suppressed the expression of fat synthesis-related factors. Specifically, to analyze the expression of peroxisome proliferator-activated receptor-gamma (PPAR-γ), RNA was extracted from the mouse epididymal fat tissue and RT-PCR was performed. The RNA was eluted from the epididymal fat using a TRI-zol solution, and then cDNA was synthesized using Maxime RT premix. The synthesized cDNA was mixed with a primer prepared by obtaining the base sequence from NCBI GenBank and a Maxime PCR premix kit, and the initial denaturation, denaturation, annealing, and extension reactions were performed at 94° C. for 5 minutes, 30 cycles and at 94° C. for 1 minute, 62° C. for 45 seconds, and 72° C. for 1 minute. Glyceraldehyde-3-phosphate (GAPDH) was used as a control gene.

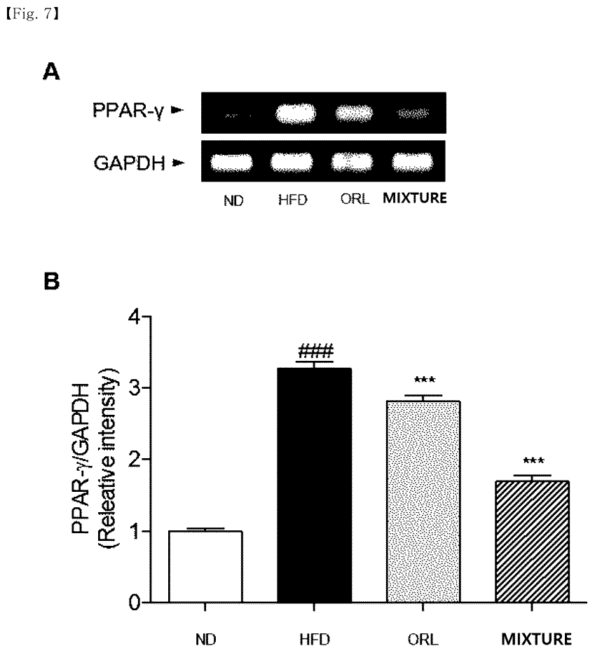

As a result, as shown in FIG. 7, it was confirmed that the expression of PPAR-γ was significantly reduced in the Formula group administered with the mixed extract of the present disclosure compared to the high-fat diet group (HFD), and it was confirmed that the expression level of PPAR-γ was reduced by 48.27% compared to the PPAR-γ expression level of the high-fat diet group.

### Example 7

### Comparison of Effects of Inhibiting Fat Accumulation of Single Extracts and Mixed Extract of Taraxacum platycarpum and Lonicerae japonica

Fat accumulation inhibitory effects of single extracts and a mixed extract of Taraxacum platycarpum and Lonicerae japonica of the present disclosure were compared. Specifically, HepG2 cells, a liver cancer cell line, were incubated in a 24-well plate at a concentration of 4×10⁵ cells/ml for 24 hours. To induce the fat accumulation, 1 mM of free fatty acid (FFA) and single extracts of Taraxacum platycarpum and Lonicerae japonica and a mixed extract (TL-700) of Taraxacum platycarpum and Lonicerae japonica were treated at a concentration of 100 μg/mL, respectively. After 24 hours, the cells were washed with cold PBS and fixed with 10% paraformaldehyde for 1 hour. The fixed cells were washed again with PBS and finally washed with 60% isopropanol. To observe the fat accumulated in the cells after washing, the cells were stained with Oil Red O (Sigma-Aldrich, St. Louis, MO, USA) dye for 20 minutes, the residual dye was removed with 60% isopropanol, and each well was observed under an optical microscope. As control groups, an untreated control group (NOR) and a group induced with only fat accumulation (FFA) were used.

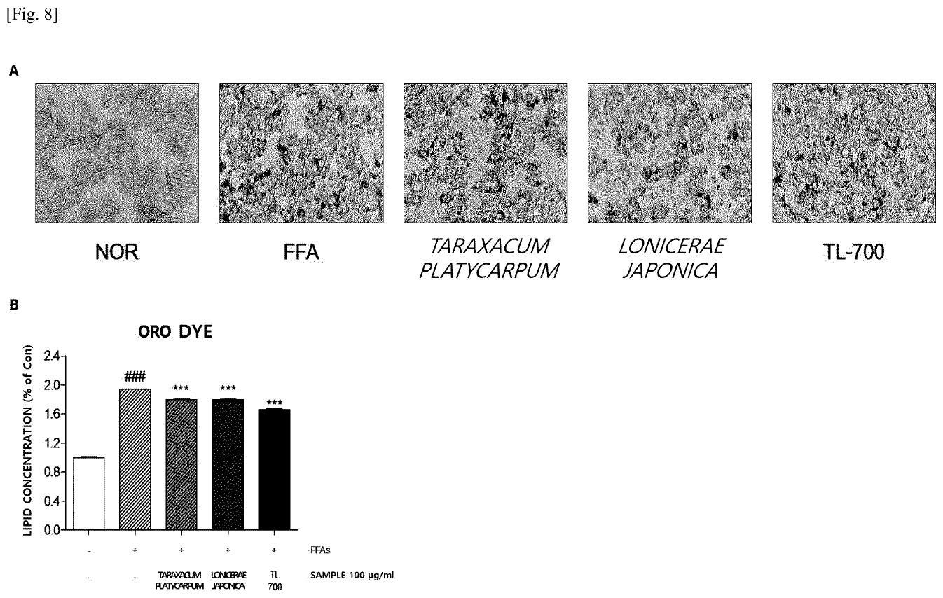

As a result, as shown in FIG. 8, compared to the NOR group, intracellular fat accumulation in the FFA group significantly increased 1.94 times, but the intracellular fat accumulation was significantly reduced by 7.2% in the Taraxacum platycarpum-alone extract group and by 7.5% in the Lonicerae japonica-alone extract group. In particular, in the group administered with the mixed extract of Taraxacum platycarpum and Lonicerae japonica, a significant synergistic effect was confirmed by decreasing the intracellular fat accumulation by 14.4%.

### <7-2>Confirmation of Regulation of Expression of Fat Metabolism Factors

A synergistic effect of the mixed extract of Taraxacum platycarpum and Lonicerae japonica of the present disclosure on regulating the expression of fat metabolism factors was confirmed. Specifically, in the cells of each group of Example 7-1, the expression of ATGL (Adipose triglyceride lipase) as a fat decomposition factor, AMPK (AMP-activated protein kinase) as a fat production inhibitor, and p-AMPK as an activated form thereof was analyzed by Western blot.

In addition, the gene expression of sterol regulatory element-binding protein-1c (SREBP-1c), which was associated with fat accumulation, and lipoprotein lipase (LPL) as a fat cell differentiation marker was analyzed by RT-PCR.

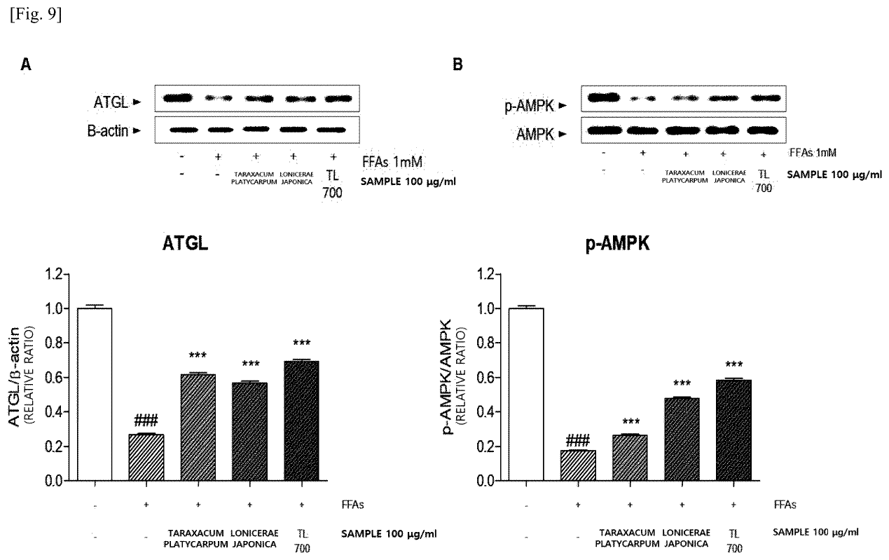

As a result, as shown in FIG. 9, compared to the NOR group, the expression of ATGL as the fat decomposition factor and AMPK as the as fat production inhibitor was significantly reduced by 73.2% and 82.5% in the FFA group. In the Taraxacum platycarpum-alone extract group, the expression level of ATGL increased by 128.9% and the expression level of AMPK increased by 50.8%. In the Lonicerae japonica-alone extract group, the expression level of ATGL increased by 111.4% and the expression level of AMPK increased by 173%. In particular, in the group administered with the mixed extract of Taraxacum platycarpum and Lonicerae japonica, the expression level of ATGL increased by 158% and the expression level of AMPK increased by 233.7% to confirm a significant synergistic effect.

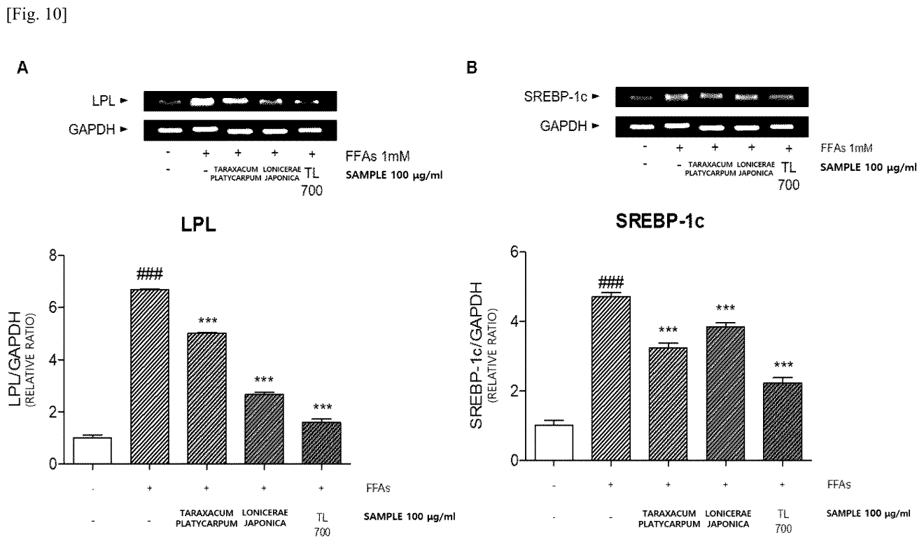

In addition, compared to the NOR group, in the FFA group, it was confirmed that the expression of SREBP-1c and LPL as the fat accumulation factors significantly increased, and was significantly decreased by administration of Taraxacum platycarpum or Lonicerae japonica, and the mixed extract (FIG. 10). In the Taraxacum platycarpum-alone extract group, the expression level of SREBP-1c decreased by 31.4% and the expression level of LPL decreased by 7.2%. In the Lonicerae japonica-alone extract group, the expression level of SREBP-1c decreased by 18.3% and the expression level of LPL decreased by 7.5%. In particular, in the group administered with the mixed extract of Taraxacum platycarpum and Lonicerae japonica, the expression level of SREBP-1c decreased by 52.8% and the expression level of LPL decreased by 14.4%. Based on these results, compared to each single extract treated group, it was confirmed that the expression of fat metabolism factors was significantly regulated by the mixed extract, and thus the synergistic effect of the mixed extract of Taraxacum platycarpum and Lonicerae japonica was confirmed.

Accordingly, it has been confirmed that the mixed extract of Taraxacum platycarpum and Lonicerae japonica of the present disclosure reduces body weight and body fat when administered to mice with obesity induced by a high-fat diet. Also, it has been confirmed that the mixed extract reduces the size of fat tissue, reduces fat vacuoles (pore) in liver tissue, reduces total cholesterol, triglycerides, and LDL-cholesterol in the blood, and increases HDL-cholesterol. Furthermore, it has been confirmed that the mixed extract suppresses the expression of PPAR-γ which is related to fat synthesis. In addition, it has been confirmed that the mixed extract has a synergistic effect on the regulation of intracellular fat accumulation and fat metabolism factors in cells in which fat accumulation is induced.

1. A method for preventing or improving obesity, comprising providing to a subject in need thereof a food composition comprising a mixed extract of Taraxacum platycarpum and Lonicerae japonica as an active ingredient.

2. The method of claim 1, wherein the Taraxacum platycarpum and the Lonicerae japonica are mixed and extracted in a weight ratio of 1 to 100:1 to 100.

3. The method of claim 1, wherein the Taraxacum platycarpum and the Lonicerae japonica are extracted with a solvent selected from the group consisting of water, lower alcohols having C1 to C4, a lower alcohol aqueous solution, hexane, chloroform, ethyl acetate, and acetone.

4. The method of claim 1, wherein the Taraxacum platycarpum and the Lonicerae japonica are mixed and extracted with a solvent in a weight ratio of 1 to 100:1 to 100.

5. The method of claim 1, wherein the subject requires reducing the body weight.

6. The method of claim 5, wherein the body weight reduction is reduction of body fat, body fat mass or body fat percentage.

7. The method of claim 1, wherein the subject requires reducing the size of fat cells within fat tissue.

8. The method of claim 1, wherein the subject requires inhibiting the formation of fat vacuoles (pore) in liver tissue.

9. The method of claim 1, wherein the subject requires reducing the content of lipids selected from the group consisting of total cholesterol, triglyceride, low density lipoprotein cholesterol (LDL-cholesterol), and very low density lipoprotein cholesterol (VLDL-cholesterol) in the blood.

10. The method of claim 1, wherein the subject requires increasing high density lipoprotein cholesterol (HDL-cholesterol) in the blood.

11. The method of claim 1, wherein the subject requires inhibiting the expression of a fat synthesis factor, peroxisome proliferator-activated receptor-gamma (PPAR-γ).

12. The method of claim 1, wherein the subject regulates regulating the expression of fat metabolism factors.

13. The method of claim 12, wherein the fat metabolism factor is selected from the group consisting of adipose triglyceride lipase (ATGL), phosphorylation-AMP-activated protein kinase (p-AMPK), lipoprotein lipase (LPL), and sterol regulatory element-binding protein-1c (SREBP-1c).

14. The method of claim 12, wherein the regulating of the expression of the fat metabolism factors is increasing the expression of ATGL or p-AMPK.

15. The method of claim 12, wherein the regulating of the expression of the fat metabolism factors is decreasing the expression of LPL or SREBP-1c.

16. A method for preventing or treating obesity, comprising administering to a subject in need thereof a pharmaceutical comprising a mixed extract of Taraxacum platycarpum and Lonicerae japonica as an active ingredient.

17. The method of claim 1, wherein the subject requires inducing body fat reduction.

18. The method of claim 16, wherein the subject requires inducing body fat reduction.

19. (canceled)
